(19) United States
(12) Patent Application Publication
(10) Pub. No.: US 2026/0223924 A1
(43) Pub. Date: Aug. 6, 2026

(54) Method for powering an electrically operated liquid atomization device with an external power source.

(71) Applicant: Vladislav Kofman, Brooklyn, NY (US)

(72) Inventor: Vladislav Kofman, Brooklyn, NY (US)

(73) Assignee: Cardinalis Group Inc., Brooklyn, NY (US)

(21) Appl. No.: 19/047,615

(22) Filed: Feb. 6, 2025

(51) Int. Cl. A24F 40/40; A24F 40/10; A61M 15/06

(52) U.S. Cl. CPC A24F 40/40; A24F 40/10; A61M 15/06; A61M 2205/8262

(57) ABSTRACT

The invention is a method of powering portable liquid atomization devices such as simulated smoking devices (e-cigarettes and vapes), or portable medical inhalation devices such as nebulizers, which do not contain an internal power source and instead can be used with and powered by a mobile device, such as a mobile smartphone (or similar device) capable of providing sufficient electric power output. It belongs to the field of portable simulated smoking devices such as vaping or e-cigarettes and portable medical inhalation devices such as nebulizers. This patent application outlines the unique aspects of the invention, pertaining specifically to the method of powering such a device, and relates to the novel operational details, e.g. connection of power and power source.

### FIELD OF INVENTION

The present disclosure relates to the technical field of electrically operated liquid atomization devices such as smoking devices (e-cigarettes and Vapes), and medical inhalation devices such as nebulizers. Specifically, the field of invention relates to the processes used for powering such devices.

### BACKGROUND

Conventional e-cigarettes, cannabinoid nebulizers, vaping devices (also known as Vapes) and portable medical nebulizers are electrically powered liquid atomization devices. These devices are battery-operated and allow users to inhale an atomized aerosol, usually containing nicotine and/or other chemicals. The atomized liquids, in the vapor form, can have various flavorings and/or medicinal purposes.

Regardless of the aerosols used, these devices have the following common characteristics, they all have a liquid holding cartridge, an atomization unit, an automatic control unit (e.g microprocessor), various sensors, and a power supply such as rechargeable batteries or single-use batteries. They usually come in a small, portable form factor, and may have either replaceable pods or refillable cartridges for the liquid to be atomized into the aerosol. The internal power source batteries are either lithium-ion (Li-ion), zinc-ion, sodium-ion, lithium-sulfur, or solid-state batteries which need to be periodically recharged, or non-rechargeable, such as alkaline batteries, which need to be periodically replaced. The single-use versions of e-cigarettes and Vapes are also available and typically use alkaline batteries.

The electrically powered liquid atomization devices unitize an internal power source to convert electric energy into heat to atomize the liquid into aerosols, using an atomizer and a set of electronic sensors. Having an internal power source constantly requires users to maintain a certain level of power within the device, for it to be operational.

Therefore, a need exists for portable personal electrically powered liquid atomization devices that do not require an internal power source. A need exists for a different power method and a device that can utilize an external power source, such as ubiquitous conventional personal mobile phones or tablets to draw power. This invention is a method of powering liquid atomization devices such as e-cigarettes, cannabinoid nebulizers, vaping devices (also known as Vapes), and portable medical nebulizers with an external power source, which addresses this need.

### DETAILED DESCRIPTION OF THE INVENTION

Described herein is the method of powering electrically powered liquid atomization devices, which atomize liquid into aerosol, in a compact and convenient form-factor designed for personal use and can either be for recreational, or medical use. This invention does not promote nicotine or cannabinoid consumption or endorse the use of nicotine or cannabinoid-based aerosols. The invention covers the power delivery method and process of using such devices with external power sources.

One embodiment is a device that is a small portable, wireless version comprising a mouthpiece, atomization unit, automatic control unit, microprocessor, various sensors, and a connector unit for power supply, such as USB-C exterior power connector, which is rotatable about the main body, or has a folding option. The device also incorporates an ergonomic portable small form factor. The handling operation of the device can be one-handed, and the user can hold the mobile phone itself while operating the device, attached to the mobile phone. The device itself does not contain an internal power source such as a rechargeable or non-rechargeable battery.

The USB connector in the current embodiment of the invention described is a USB-C® utilizing a USB Type-C® connection and the corresponding protocol for power delivery, other embodiments may use similar Universal Serial Bus (USB) power delivery protocols but via different USB connectors; similarly, this is applicable to older or newer versions or the connector. The sole purpose of the plug is to connect the device to the external power source, such as a mobile phone supporting the power output required, and hold it in place.

It is accordingly the goal of this detailed description of the invention is to describe and cover the method for powering such as a device having all of the advantages and benefits set forth above and described in greater detail hereinafter in this application.

The current embodiment of the invention has one atomization implementation through a function of conversion of liquid to an aerosol, using sensors that detect air flow usually from the inhale process, and a heating coil within the device, with the main differentiator from the other devices currently in existence, in that no internal power source such as rechargeable batteries, or any kind of chemically based batteries are required. In one embodiment of the invention, the rigid structure enclosing the atomization unit, automatic control unit, microprocessor, various sensors (such as airflow sensor), relay electronics, required wiring, and control module may be formed of molded plastic, metal, or similar rigid materials, and would be completely sealed and waterproof. The mouthpiece and the power connector parts can also be molded from high-grade plastic or hypoallergic stainless steel or similar metal alloy, or other rigid materials. The mouthpiece is detachable or removable for replacement or cleaning operation by the users, as needed.

In the current embodiment of the invention, the device does not have an on/off button (used to turn on the device). Instead, the action of plugging or unplugging the device to or from the mobile power source (such as a mobile phone or similar power device) itself is equivalent to turning the device On or Off, without the need for the user to press any buttons.

This invention and description also comprise other capabilities related to this type of device and power method, without an internal power source, as may later fully appear and which are inherently possessed by the invention. The one embodiment described herein is mainly applicable to the electric simulated smoking devices (e-cigarettes and Vapes), and medical inhalation devices such as portable nebulizers, but other applications are possible.

### DESCRIPTION OF THE DRAWINGS

While the accompanying drawings demonstrate the preferred embodiment of the invention, it should be understood that the same is susceptible to modification and change without departing from the spirit of the invention, such as a change in the shape, or power delivery protocol.

The drawings are not to exact scale. The drawings are not intended to portray specific sizes or parameters of the parts of the invention and should be considered merely representations. The drawings are intended to depict example embodiments of the invention, and therefore should not be considered as limiting in scope, but represent the essence of the invention. In the drawings, the same numbering may represent like elements. Furthermore, certain elements in some of the figures may be not labeled due to overlay with other depicted elements, or depicted not-to-scale, for explanatory clarity only.

FIG. 1 is a complete exterior and sample interior view of the embodiment of the invention. Showing the device in its entirety, and depicts the mouth piece which can be detached for cleaning, the part enclosing the cartridge (which can hold atomization liquid), a heater to vaporize the liquid, various sensors and microprocessors, and wiring to the power USB connector. The power connector is on a hinged module which is rotatable in relation to the main body.

FIG. 2 is a complete exterior and sample interior view of the embodiment of the invention. The diagram is mainly the same as FIG. 2, showing the device in its entirety, and depicts the mouthpiece which can be detached for cleaning, the part enclosing the cartridge (which can hold atomization liquid), the heater coil to vaporize the liquid, various sensors and microprocessors and wiring to the power USB connector. However, the power connector on a hinged module is shown rotated at 90 degrees about the main body. Connected to the exterior power, this position allows for simultaneous operation of the device and the mobile phone.

FIG. 3 is a detailed view of the power connector part, rotated at 45 degrees, depicting a side view of the connector.

### DESCRIPTION OF THE PREFERRED EMBODIMENT

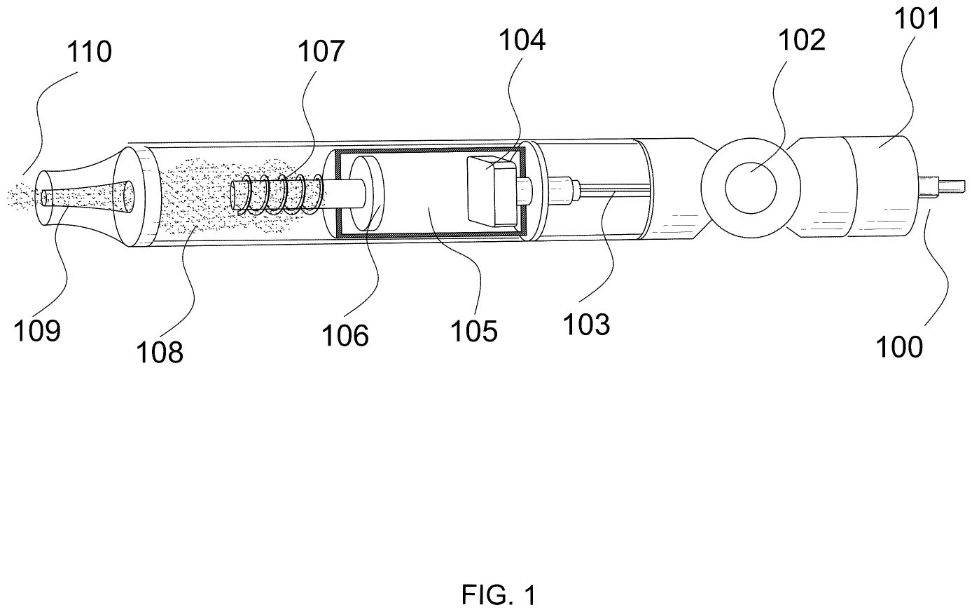

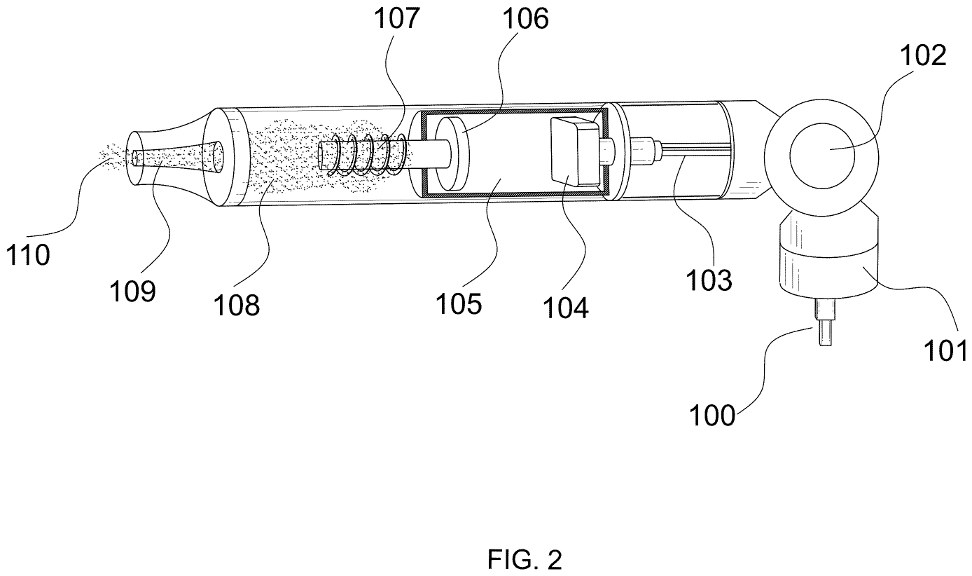

Referring to FIG. 1 and FIG. 2 of the drawings is a device, which works by using electric energy to heat the coil 107 and vaporize aerosol liquid 108, released through mouthpiece 109, as vapor 110. The electric energy, however, in the current embodiment, is not stored in the device but instead flows from the power connector 100 which is connected to the rotating part 101, attached by hinge 102, to the main body 105. The integrated power plug or connector 100, is protruding for a convenient way to connect to the external power source.

The parts enclosing the liquid holding cartridge 108, heater coil 107 to vaporize the liquid, air flow sensor 106 which detects inhale action, microprocessor 104 which controls heater coil 107, wiring 103, hinge 102, and connector 101, are molded from a rigid material, such as metal or hard plastic. The connector 100 is compatible with separate power sources, such as, but not limited to USB (e.g. USB-C®) connectors.

In the preferred embodiment, the device is controlled by the microprocessor 104, without an on/off switch, instead activated by inhale action. The draw of power from the external power source is consistent and is not affected by the external mobile power source battery levels or remaining battery life. The device is designed to work for as long as an external power source can deliver power.

The connector (power plug) 100 protruding from the bottom section of the device 101, is plugged into the mobile phone and securely holds the entire device in place, regardless of the rotational alignment. The connection is via the industry standard USB connector.

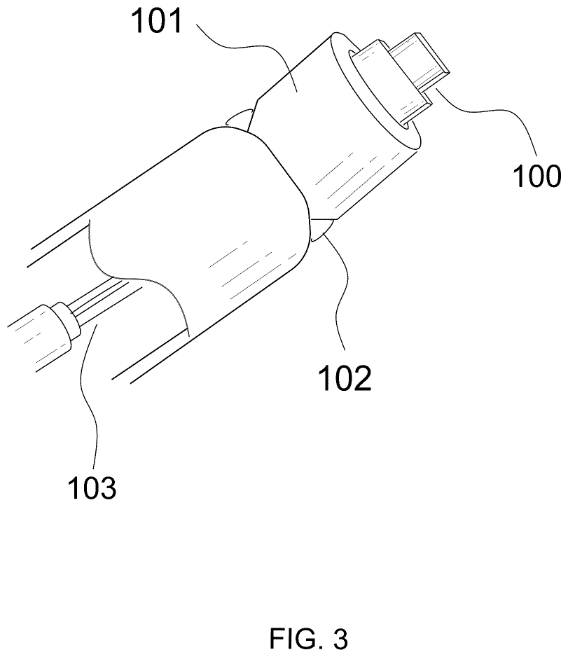

Referring to FIG. 3 of the drawings, the power connector part 101 is rotated on the hinged 102 and attached to the internal components of the main body by required wiring 103. The USB connection 100 is visible in more detail.

The current illustrations are intended to represent the one embodiment of the invention without departing from the spirit, scope, and purpose of the invention. Thus, it is intended that the present invention cover the modifications and variations of this invention provided they come within the scope of the appended claims and their equivalents.

The method for powering electric e-cigarettes (or Vaping devices) described herein offers a convenient, and user-friendly solution, without the need to change or replace batteries. The invention combines portability with features that address common drawbacks of existing electric-simulated smoking devices (e-cigarettes and Vapes), and portable medical inhalation devices (nebulizers). The invention's unique features make it an ideal method of powering such devices. The compact size and battery-less operated functionality also make this approach ideal for travel and on-the-go use. Reference will now be made to the claims of the invention.

1. A method of powering electrically operated liquid atomization devices such as simulated smoking devices (e-cigarettes and vapes), and portable medical inhalation devices such as portable nebulizers, with an exterior power source that has sufficient power for the atomization process.

2. A portable electrically operated device, with no independent internal power source, powered via an external power source, such as a mobile smartphone.

3. The cordless operation, of portable electrically operated device of claim 2, by virtue of being attached to draw power from an external power source, such as a mobile phone.

4. The liquid atomization device of claim 2, provides unlimited continuous usage for as long as the external power source allows.

5. The liquid atomization device of claim 2, with chambers for storage of vapor-producing liquid, air flow detection sensors, atomization heating coils, a control unit such as a microprocessor, other required sensors, and a power connection module.
